(19) United States
(12) Patent Application Publication
(10) Pub. No.: US 2022/0126031 A1
(43) Pub. Date: Apr. 28, 2022

(54) Steam Therapy Assembly

(71) Applicant: Rashawn Thurman, Calumet City, IL (US)

(72) Inventor: Rashawn Thurman, Calumet City, IL (US)

(21) Appl. No.: 17/080,356

(22) Filed: Oct. 26, 2020

(51) Int. Cl. A61M 15/00; A61H 33/06; A61M 16/16; F24H 3/00; H05B 1/02

(52) U.S. Cl. CPC A61M 15/0003; A61H 33/065; A61M 16/162; F24H 3/002; H05B 1/0297; A61H 2201/105; A61M 2205/583; A61M 2205/3584; A61M 2205/502; A61M 2209/10; A61M 2205/8206; A61H 2033/068

(57) ABSTRACT

A steam therapy assembly includes a housing that has an intake and an exhaust to pass air through the intake and the exhaust. A water tank is removably attachable to the housing and the water tank has a fluid port to pass water therethrough for filling the water tank with water. An oil cartridge contains oil and the oil cartridge is removably insertable into the housing. A vaporizing unit is integrated into the housing and the vaporizing unit is in fluid communication between the intake and the exhaust to urge air into the intake and outwardly through the exhaust when the vaporizing unit is turned on. The vaporizing unit vaporizes the water in the water tank into a steam. Additionally, the vaporizing unit vaporizes the oil in the oil cartridge thereby mixing the vaporized oil with the vaporized water.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to therapy devices and more particularly pertains to a new therapy device for producing a therapeutic steam for treating symptoms of a respiratory infection.

### (2) Description of Related Art Including Information Disclosed Under 37 CFR 1.97 and 1.98

The prior art relates to therapy devices including a variety of aromatherapy devices that are integrated into a bathroom shower. The prior art discloses a variety of aromatherapy devices that each can produce water vapor, oil vapor or a combination of both for aromatherapy. The prior art also discloses a variety of aromatherapy devices that are integrated into steam room or sauna.

### BRIEF SUMMARY OF THE INVENTION

An embodiment of the disclosure meets the needs presented above by generally comprising a housing that has an intake and an exhaust to pass air through the intake and the exhaust. A water tank is removably attachable to the housing and the water tank has a fluid port to pass water therethrough for filling the water tank with water. An oil cartridge contains oil and the oil cartridge is removably insertable into the housing. A vaporizing unit is integrated into the housing and the vaporizing unit is in fluid communication between the intake and the exhaust to urge air into the intake and outwardly through the exhaust when the vaporizing unit is turned on. The vaporizing unit vaporizes the water in the water tank into a steam. Additionally, the vaporizing unit vaporizes the oil in the oil cartridge thereby mixing the vaporized oil with the vaporized water.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a steam therapy assembly according to an embodiment of the disclosure.

FIG. 2 is a front view of an embodiment of the disclosure.

FIG. 3 is a front exploded view of an embodiment of the disclosure.

FIG. 4 is a bottom view of an embodiment of the disclosure.

FIG. 5 is a right side phantom view of an embodiment of the disclosure.

FIG. 6 is a perspective view of a remote control of an embodiment of the disclosure.

FIG. 7 is a schematic view of an embodiment of the disclosure.

### DETAILED DESCRIPTION OF THE INVENTION

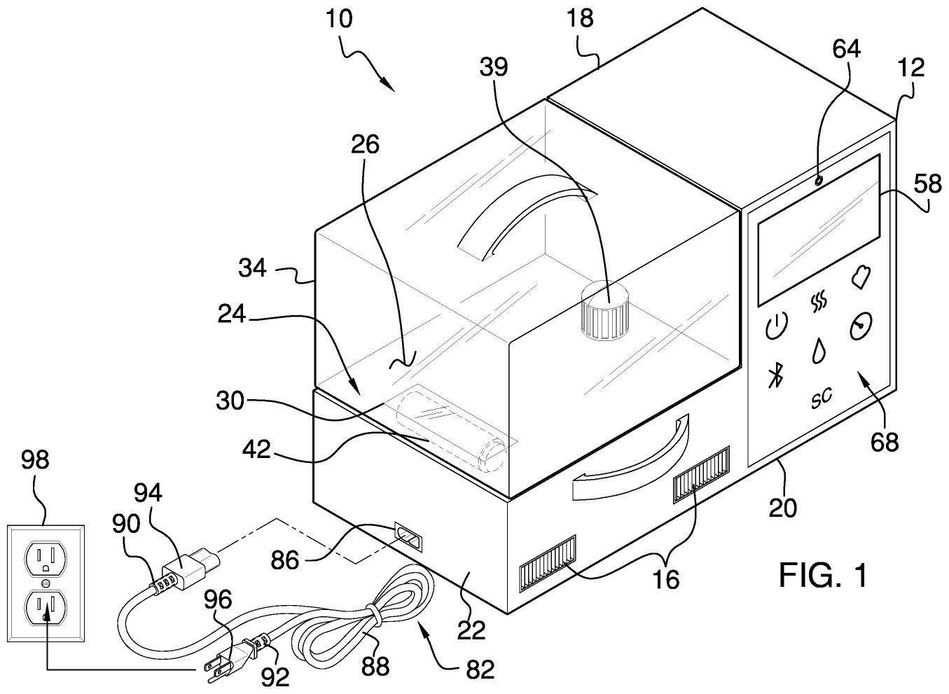

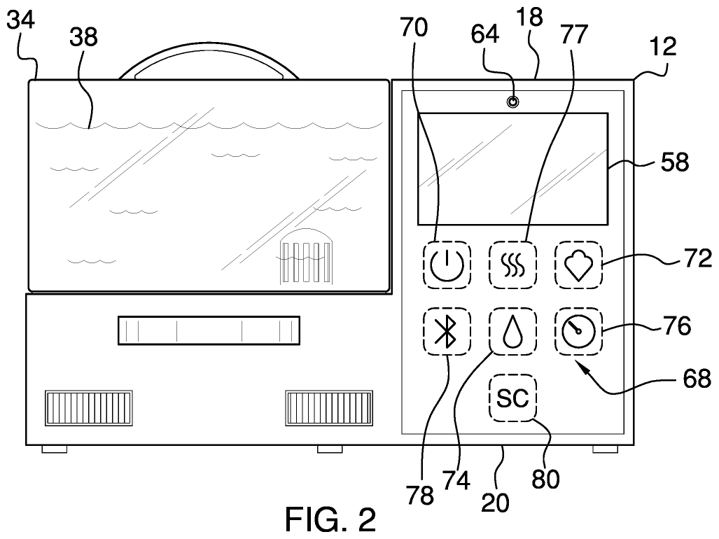

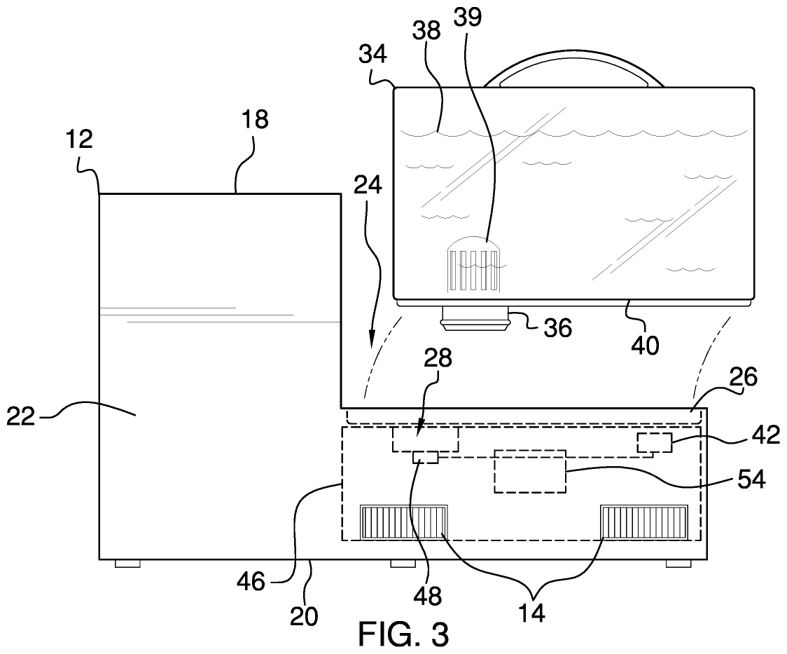

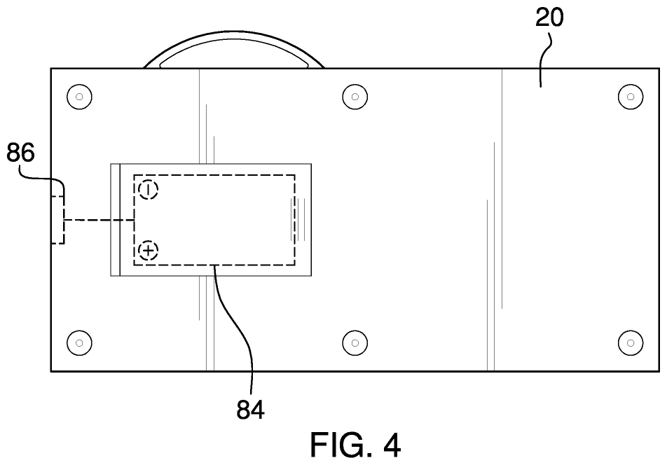

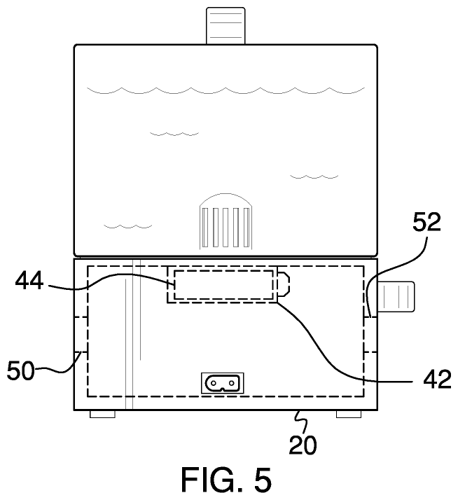

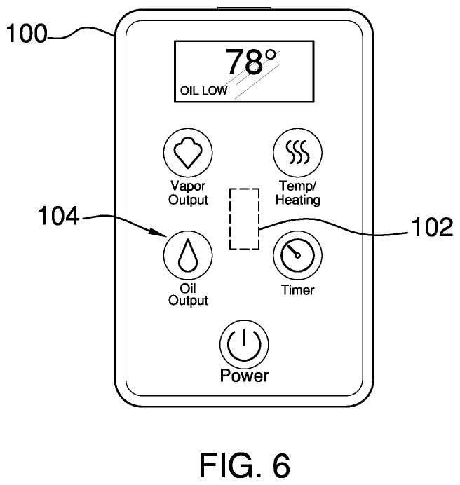

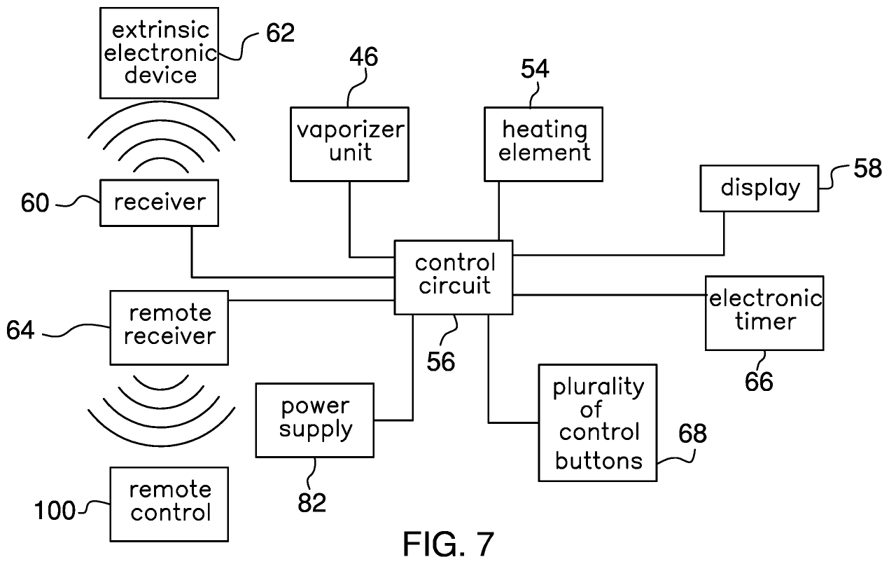

With reference now to the drawings, and in particular to FIGS. 1 through 7 thereof, a new therapy device embodying the principles and concepts of an embodiment of the disclosure and generally designated by the reference numeral 10 will be described.

As best illustrated in FIGS. 1 through 7, the steam therapy assembly 10 generally comprises a housing 12 that has an intake 14 and an exhaust 16 each being integrated therein to pass air through the intake 14 and the exhaust 16. The housing 12 has a top wall 18, a bottom wall 20 and an outer wall 22 extending therebetween. The top wall 18 has a recessed portion 24 extending toward the bottom wall 20, and the recessed portion 24 has an upper bounding surface 26. The upper bounding surface 26 has a fluid inlet 28 extending therethrough into an interior of the housing 12. Additionally, the upper bounding surface 26 has a cartridge well 30 extending downwardly toward the bottom wall 20.

A water tank 34 is provided for containing water and the water tank 34 is removably attachable to the housing 12. The water tank 34 has a fluid port 36 that is fluidly coupled thereto for passing water therethrough to fill the water tank 34 with water 38. The water tank 34 is positionable in the recessed portion 24 of the top wall 18 of the housing 12 and the water tank 34 has a lower wall 40. The fluid port 36 extends downwardly from the lower wall 40. Moreover, the fluid port 36 engages the fluid inlet 28 in the upper bounding surface 26 of the recessed portion 24 to pass the water 38 into the housing 12. The fluid port 36 includes a filter 39 that is fluidly coupled thereto for filtering particles from the water passing through the fluid port 36. A handle 41 is attached to the water tank 34 to facilitate the water tank 34 to be carried and the water tank 34 may be comprised of a translucent material thereby facilitating the water 38 in the water tank 34 to be visible.

An oil cartridge 42 is provided that contains oil 44 and the oil cartridge 42 is removably insertable into the housing 12. The oil cartridge 42 is positionable into the cartridge well 30 in the upper bounding surface 26 of the recessed portion 24 of the top wall 18 of the housing 12. In this way the oil cartridge 42 can release the oil 44 into the housing 12. The oil 44 may be an essential oil, such as lavender oil or the like. The oil 44 may be a medicinal oil, such as cannabidiol or the like. Moreover, the oil 44 may be any non-toxic oil that is appropriate for human consumption.

A vaporizing unit 46 is provided and the vaporizing unit 46 is integrated into the housing 12. The vaporizing unit 46 is in fluid communication between the intake 14 and the exhaust 16 to urge air into the intake 14 and outwardly through the exhaust 16 when the vaporizing unit 46 is turned on. In this respect the vaporizing unit 46 may include an electric motor and a fan that is rotatably coupled to the motor for blowing air. The vaporizing unit 46 is in fluid communication with the water tank 34 when the water tank 34 is removably attached to the housing 12. In this way the vaporizing unit 46 can vaporize the water in the water tank 34 into a steam is urged outwardly through the exhaust 16. Moreover, the vaporizing unit 46 is in fluid communication with the oil cartridge 42 when the oil cartridge 42 is inserted into the housing 12 to vaporize the oil in the oil cartridge 42 thereby mixing the vaporized oil with the vaporized water. In this respect the vaporizing unit 46 may include an ultrasonic humidifier or other similar electronic device that is capable of vaporizing liquid into an inhalable steam.

The vaporizing unit 46 has a vapor input 48, an air input 50 and an air outlet 52. The vapor input 48 is fluidly coupled to each of the fluid inlet 28 in the housing 12 and the cartridge well 30 in the housing 12. The air input 50 is fluidly coupled to the intake 14 in the housing 12 and the air outlet 52 is fluidly coupled to the exhaust 16 in the housing 12. Additionally, the vapor input 48 is in fluid communication with the air outlet 52. The vaporizing unit 46 includes a heating element 54 that is in thermal communication with the air outlet 52 to heat the vaporized water and the vaporized oil when the heating element 54 is turned on. The heating element 54 may comprise an electric heating element that has an operational temperature ranging between approximately 120.0 degrees Fahrenheit and 150.0 degrees Fahrenheit.

A control circuit 56 is integrated into the housing 12 and the control circuit 56 is electrically coupled to the vaporizing unit 46 and the heating element 54. The control circuit 56 receives a water input, an oil input, a heat input, a timer input and an off input. The vaporizing unit 46 is turned on when the control circuit 56 receives the on input and the vaporizing unit 46 vaporizes the water 38 when the control circuit 56 receives the water input. Moreover, the vaporizing unit 46 vaporizes the oil 44 when the control circuit 56 receives the oil input. The heating element 54 is turned on when the control circuit 56 receives the heat input and the vaporizing unit 46 is turned off when the control circuit 56 receives the off input.

A display 58 is coupled to the outer wall 22 of the housing 12 such that the display 58 is visible to a user. The display 58 is electrically coupled to the control circuit 56. Additionally, the display 58 displays indicia comprising various operational parameters of the control circuit 56 thereby facilitating the display 58 to visually communicate operational status of the vaporizing unit 46 and the heating element 54. The display 58 may comprise an electronic display such as an LCD or other similar electronic device.

A receiver 60 is integrated into the housing 12 and the receiver 60 is electrically coupled to the control circuit 56. The receiver 60 is in wireless communication with an extrinsic electronic device 62 such that the control circuit 56 can be remotely controlled by the extrinsic electronic device 62. The receiver 60 may be a radio frequency receiver or the like and the receiver 60 may employ Bluetooth communication protocols. The extrinsic electronic device 62 may be a smart phone, or other similar device, that has wireless communication capabilities and which employs Bluetooth communication protocols. A remote receiver 64 is coupled to the outer wall 22 of the housing 12 and the remote receiver 64 is electrically coupled to the control circuit 56. The remote receiver 64 may comprise a radio frequency receiver, an infra-red light receiver or any other receiver commonly employed for remote control 100s.

An electronic timer 66 is integrated into the housing 12 and the electronic timer 66 is electrically coupled to the control circuit 56. The electronic timer 66 is turned on when the control circuit 56 receives the timer input and the electronic timer 66 counts down a pre-determined duration of time when the electronic timer 66 is turned on. Additionally, the control circuit 56 receives the off input when the electronic timer 66 finishes counting down the pre-determined duration of time. The pre-determined duration of time may range between approximately 5.0 minutes and 20.0 minutes.

A plurality of control buttons 68 is each integrated into the outer wall 22 of the housing 12 such that each of the control buttons 68 can be manipulated by a user. The plurality of control buttons 68 includes a power button 70, a water button 72, an oil button 74, a timer button 76, a heat button 77, a Bluetooth button 78 and a self clean button 80. The control circuit 56 receives the on input when the power button 70 is initially depressed and the control circuit 56 receives the off input when the power button 70 is subsequently depressed. The control circuit 56 receives the water input when the water button 72 is depressed, the control circuit 56 receives the oil input when the oil button 74 is depressed and the control circuit 56 receives the timer input when the timer button 76 is depressed. Moreover, the control circuit 56 commences a cleaning sequence when the self clean button 80 is depressed thereby facilitating the vaporizing unit 46 to perform a predetermined self cleaning process. The receiver 60 is actuated to communicate with the extrinsic electronic device 62 when the Bluetooth button 78 is depressed. The control circuit 56 receives a heat input when the heat button 77 is depressed and the heating element 54 is turned on when the control circuit 56 receives the heat input.

A power supply 82 is integrated into the housing 12 and the power supply 82 is electrically coupled to the control circuit 56. The power supply 82 includes a rechargeable battery 84 that is positioned in the housing 12 and the rechargeable battery 84 is electrically coupled to the control circuit 56. The power supply 82 includes a power port 86 that is recessed into the outer wall 22 of the housing 12 and the power port 86 is electrically coupled to the rechargeable battery 84. The power supply 82 additionally includes a power cord 88 that has a first end 90 and a second end 92. A female plug 94 is electrically coupled to the first end 90 and the female plug 94 electrically engages the power port 86. A male plug 96 is electrically coupled to the second end 92 and the male plug 96 can be electrically coupled to a power source 98 comprising a female electrical outlet for charging the rechargeable battery 84.

A remote control 100 is in wireless communication with the vaporizing unit 46 thereby facilitating the vaporizing unit 46 to be remotely controlled. The remote control 100 includes a transmitter 102 that is in wireless communication with the remote receiver 64 on the housing 12. The remote control 100 includes a plurality of control buttons 104, and the remote control 100 broadcasts a water command, an oil command, a heat command, a timer command and an off command when respective ones of the control buttons 104 on the remote control 100 is depressed. The control circuit 56 receives the water input, the oil input, the heat input, the timer input and the off input when the remote receiver 64 receives a corresponding one of the water command, the oil command, the heat command, the timer command or the off command.

In use, the water tank 34 is filled with water, the oil cartridge 42 is positioned in the cartridge well 30 and the water tank 34 is positioned in the recessed portion 24 of the top wall 18 of the housing 12. The control buttons 68 are manipulated to produce steam of either water, the oil or a combination of both. Additionally, the housing 12 can be positioned in a bathroom while a shower is being employed to produce steam. In this way the bathroom can be filled with steam from the shower and with the steam from the vaporizing unit 46. Thus, a user can relieve symptoms of a respiratory infection by inhaling the mixture of water vapor and oil vapor that is produced in the bathroom. Moreover, the vaporizing unit 46 can be remotely controlled or the vaporizing unit 46 can be controlled with the control buttons 68.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A steam therapy assembly for producing a steam comprised of water vapor and an oil vapor for treating symptoms of respiratory infection, said assembly comprising:
a housing having an intake and an exhaust each being integrated therein wherein said housing is configured to pass air through said intake and said exhaust;
a water tank being configured to contain water, said water tank being removably attachable to said housing, said water tank having a fluid port being fluidly coupled thereto wherein said fluid port is configured to pass water therethrough for filling said water tank with water;
an oil cartridge being configured to contain an oil, said oil cartridge being removably insertable into said housing;
a vaporizing unit being integrated into said housing, said vaporizing unit being in fluid communication between said intake and said exhaust wherein said vaporizing unit is configured to urge air into said intake and outwardly through said exhaust when said vaporizing unit is turned on, said vaporizing unit being in fluid communication with said water tank when said water tank is removably attached to said housing wherein said vaporizing unit is configured to vaporize the water in said water tank into a steam being urged outwardly through said exhaust, said vaporizing unit being in fluid communication with said oil cartridge when said oil cartridge is inserted into said housing wherein said vaporizing unit is configured to vaporize the oil in said oil cartridge thereby mixing the vaporized oil with the vaporized water; and
a remote control being in wireless communication with said vaporizing unit thereby facilitating said vaporizing unit to be remotely controlled.

2. The assembly according to claim 1, wherein said housing has a top wall, a bottom wall and an outer wall extending therebetween, said top wall having a recessed portion extending toward said bottom wall, said recessed portion having an upper bounding surface, said upper bounding surface having a fluid inlet extending therethrough into an interior of said housing, said upper bounding surface having a cartridge well extending downwardly toward said bottom wall, said intake extending through said outer wall.

3. The assembly according to claim 2, wherein said water tank is positionable in said recessed portion of said top wall of said housing, said water tank having a lower wall, said fluid port extending downwardly from said lower wall, said fluid port engaging said fluid inlet in said upper bounding surface of said recessed portion wherein said fluid port is configured to pass the water into said housing.

4. The assembly according to claim 2, wherein said oil cartridge is positionable into said cartridge well in said upper bounding surface of said recessed portion of said top wall of said housing wherein said oil cartridge is configured to release the oil into said housing.

5. The assembly according to claim 2, wherein said vaporizing unit has a vapor input, an air input and an air outlet, said vapor input being fluidly coupled to each of said fluid inlet in said housing and said cartridge well in said housing, said air input being fluidly coupled to said intake in said housing, said air outlet being fluidly coupled to said exhaust in said housing, said vapor input being in fluid communication with said air outlet.

6. The assembly according to claim 5, wherein said vaporizing unit includes a heating element being in thermal communication with said air outlet wherein said heating element is configured to heat the vaporized water and the vaporized oil when said heating element is turned on.

7. The assembly according to claim 6, further comprising a control circuit being integrated into said housing, said control circuit being electrically coupled to said vaporizing unit and said heating element, said control circuit receiving an water input, said control circuit receiving an oil input, said control circuit receiving a heat input, said control circuit receiving a timer input, said control circuit receiving an off input.

8. The assembly according to claim 7, wherein said vaporizing unit is turned on when said control circuit receives said on input, said vaporizing unit vaporizing the water when said control circuit receives said water input, said vaporizing unit vaporizing the oil when said control circuit receives said oil input, said heating element being turned on when said control circuit receives said heat input, said vaporizing unit being turned off when said control circuit receives said off input.

9. The assembly according to claim 7, further comprising a display being coupled to said outer wall of said housing wherein said display is configured to be visible to a user, said display being electrically coupled to said control circuit, said display displaying indicia comprising various operational parameters of said control circuit thereby facilitating said display to visually communicate operational status of said vaporizing unit and said heating element.

10. The assembly according to claim 7, further comprising a receiver being integrated into said housing, said receiver being electrically coupled to said control circuit, said receiver being in wireless communication with an extrinsic electronic device wherein said control circuit is configured to be remotely controlled by the extrinsic electronic device.

11. The assembly according to claim 7, further comprising a remote receiver being coupled to said outer wall of said housing, said remote receiver being electrically coupled to said control circuit.

12. The assembly according to claim 7, further comprising an electronic timer being integrated into said housing, said electronic timer being electrically coupled to said control circuit, said electronic timer being turned on when said control circuit receives said timer input, said electronic timer counting down a pre-determined duration of time when said electronic timer is turned on, said control circuit receiving said off input when said electronic timer finishes counting down said pre-determined duration of time.

13. The assembly according to claim 7, wherein:
said assembly includes a plurality of control buttons, each of said control buttons being integrated into said outer wall of said housing wherein each of said control buttons is configured to be manipulated by a user, said plurality of control buttons including a power button, a water button, an oil button, a timer button, a Bluetooth button and a self clean button;
said control circuit receives said on input when said power button is initially depressed, said control circuit receiving said off input when said power button is subsequently depressed, said control circuit receiving said water input when said water button is depressed, said control circuit receiving said oil input when said oil button is depressed, said control circuit receiving said timer input when said timer button is depressed;
said control circuit commences a cleaning sequence when said self clean button is depressed thereby facilitating said vaporizing unit to perform a predetermined self cleaning process; and
said receiver is actuated to communicate with an extrinsic electronic device when said Bluetooth button is depressed.

14. The assembly according to claim 7, further comprising a power supply being integrated into said housing, said power supply being electrically coupled to said control circuit, said power supply comprising:
a rechargeable battery being positioned in said housing, said rechargeable battery being electrically coupled to said control circuit;
a power port being recessed into said outer wall of said housing, said power port being electrically coupled to said rechargeable battery; and
a power cord having a first end and a second end, said first end having a female plug being electrically coupled thereto, said female plug electrically engaging said power port, said second end having a male plug being electrically coupled thereto wherein said male plug is configured to be electrically coupled to a power source comprising a female electrical outlet for charging said rechargeable battery.

15. The assembly according to claim 11, wherein said remote control includes a transmitter being in wireless communication with said remote receiver on said housing, said remote control including a plurality of control buttons, said remote control broadcasting a water command, an oil command, a heat command, a timer command, and an off command when respective ones of said control buttons is depressed, said control circuit receiving said water input, said oil input, said heat input, said timer input and said off input when said remote receiver receives a corresponding one of said water command, said oil command, said heat command, said timer command or said off command.

16. A steam therapy assembly for producing a steam comprised of water vapor and an oil vapor for treating symptoms of respiratory infection, said assembly comprising:
a housing having an intake and an exhaust each being integrated therein wherein said housing is configured to pass air through said intake and said exhaust, said housing having a top wall, a bottom wall and an outer wall extending therebetween, said top wall having a recessed portion extending toward said bottom wall, said recessed portion having an upper bounding surface, said upper bounding surface having a fluid inlet extending therethrough into an interior of said housing, said upper bounding surface having a cartridge well extending downwardly toward said bottom wall, said intake extending through said outer wall;
a water tank being configured to contain water, said water tank being removably attachable to said housing, said water tank having a fluid port being fluidly coupled thereto wherein said fluid port is configured to pass water therethrough for filling said water tank with water, said water tank being positionable in said recessed portion of said top wall of said housing, said water tank having a lower wall, said fluid port extending downwardly from said lower wall, said fluid port engaging said fluid inlet in said upper bounding surface of said recessed portion wherein said fluid port is configured to pass the water into said housing;
an oil cartridge being configured to contain an oil, said oil cartridge being removably insertable into said housing, said oil cartridge being positionable into said cartridge well in said upper bounding surface of said recessed portion of said top wall of said housing wherein said oil cartridge is configured to release the oil into said housing;
a vaporizing unit being integrated into said housing, said vaporizing unit being in fluid communication between said intake and said exhaust wherein said vaporizing unit is configured to urge air into said intake and outwardly through said exhaust when said vaporizing unit is turned on, said vaporizing unit being in fluid communication with said water tank when said water tank is removably attached to said housing wherein said vaporizing unit is configured to vaporize the water in said water tank into a steam being urged outwardly through said exhaust, said vaporizing unit being in fluid communication with said oil cartridge when said oil cartridge is inserted into said housing wherein said vaporizing unit is configured to vaporize the oil in said oil cartridge thereby mixing the vaporized oil with the vaporized water, said vaporizing unit having a vapor input, an air input and an air outlet, said vapor input being fluidly coupled to each of said fluid inlet in said housing and said cartridge well in said housing, said air input being fluidly coupled to said intake in said housing, said air outlet being fluidly coupled to said exhaust in said housing, said vapor input being in fluid communication with said air outlet, said vaporizing unit including a heating element being in thermal communication with said air outlet wherein said heating element is configured to heat the vaporized water and the vaporized oil when said heating element is turned on;
a control circuit being integrated into said housing, said control circuit being electrically coupled to said vaporizing unit and said heating element, said control circuit receiving an water input, said control circuit receiving an oil input, said control circuit receiving a heat input, said control circuit receiving a timer input, said control circuit receiving an off input, said vaporizing unit being turned on when said control circuit receives said on input, said vaporizing unit vaporizing the water when said control circuit receives said water input, said vaporizing unit vaporizing the oil when said control circuit receives said oil input, said heating element being turned on when said control circuit receives said heat input, said vaporizing unit being turned off when said control circuit receives said off input;
a display being coupled to said outer wall of said housing wherein said display is configured to be visible to a user, said display being electrically coupled to said control circuit, said display displaying indicia comprising various operational parameters of said control circuit thereby facilitating said display to visually communicate operational status of said vaporizing unit and said heating element;
a receiver being integrated into said housing, said receiver being electrically coupled to said control circuit, said receiver being in wireless communication with an extrinsic electronic device wherein said control circuit is configured to be remotely controlled by the extrinsic electronic device;
a remote receiver being coupled to said outer wall of said housing, said remote receiver being electrically coupled to said control circuit;
an electronic timer being integrated into said housing, said electronic timer being electrically coupled to said control circuit, said electronic timer being turned on when said control circuit receives said timer input, said electronic timer counting down a pre-determined duration of time when said electronic timer is turned on, said control circuit receiving said off input when said electronic timer finishes counting down said pre-determined duration of time;
a plurality of control buttons, each of said control buttons being integrated into said outer wall of said housing wherein each of said control buttons is configured to be manipulated by a user, said plurality of control buttons including a power button, a water button, an oil button, a timer button, a Bluetooth button and a self clean button, said control circuit receiving said on input when said power button is initially depressed, said control circuit receiving said off input when said power button is subsequently depressed, said control circuit receiving said water input when said water button is depressed, said control circuit receiving said oil input when said oil button is depressed, said control circuit receiving said timer input when said timer button is depressed, said control circuit commencing a cleaning sequence when said self clean button is depressed thereby facilitating said vaporizing unit to perform a predetermined self cleaning process, said receiver being actuated to communicate with an extrinsic electronic device when said Bluetooth button is depressed;
a power supply being integrated into said housing, said power supply being electrically coupled to said control circuit, said power supply comprising:
a rechargeable battery being positioned in said housing, said rechargeable battery being electrically coupled to said control circuit;
a power port being recessed into said outer wall of said housing, said power port being electrically coupled to said rechargeable battery; and
a power cord having a first end and a second end, said first end having a female plug being electrically coupled thereto, said female plug electrically engaging said power port, said second end having a male plug being electrically coupled thereto wherein said male plug is configured to be electrically coupled to a power source comprising a female electrical outlet for charging said rechargeable battery; and
a remote control being in wireless communication with said vaporizing unit thereby facilitating said vaporizing unit to be remotely controlled, said remote control including a transmitter being in wireless communication with said remote receiver on said housing, said remote control including a plurality of control buttons, said remote control broadcasting a water command, an oil command, a heat command, a timer command, and an off command when respective ones of said control buttons is depressed, said control circuit receiving said water input, said oil input, said heat input, said timer input and said off input when said remote receiver receives a corresponding one of said water command, said oil command, said heat command, said timer command or said off command.
